(19) United States
(12) Patent Application Publication
(10) Pub. No.: US 2022/0325222 A1
(43) Pub. Date: Oct. 13, 2022

(54) LABORATORY DEVICE WITH LOW PARTICLE EMISSION

(71) Applicant: Thermo Electron LED GmbH, Langenselbold (DE)

(72) Inventors: Rainer Schuck, Kahl am Main (DE); Juergen Schneider, Freigericht (DE); Simone Schaefer, Gelnhausen (DE); Tino Barnkoth, Moembris (DE)

(21) Appl. No.: 17/675,528

(22) Filed: Feb. 18, 2022

Related U.S. Application Data
(60) Provisional application No. 63/183,779, filed on May 4, 2021.

(30) Foreign Application Priority Data
Apr. 9, 2021 (DE) 10 2021 108 910.7

(51) Int. Cl. C12M 1/42; C12M 1/00; C12M 1/34; C12M 1/36

(52) U.S. Cl. CPC C12M 35/04; C12M 23/20; C12M 23/34; C12M 29/04; C12M 29/20; C12M 41/40; C12M 41/48

(57) ABSTRACT

The present invention relates to a laboratory device, wherein the laboratory device has an outer housing which defines an interior of the device, wherein the laboratory device is designed to assume an operating state at which a pressure in the interior of the device is lower than an ambient pressure in the environment of the laboratory device. The present invention also relates to the use of the laboratory device in a clean room.

### CROSS-REFERENCE TO RELATED APPLICATION

The present application claims priority under 35 U.S.C. § 119 of German Patent Application No. 10 2021 108 910.7, filed Apr. 9, 2021, and claims the filing benefit of U.S. Provisional Application Ser. No. 63/183,779, filed May 4, 2021, the disclosures of which are incorporated herein by reference in their entireties.

### FIELD OF THE INVENTION

The present invention relates to a laboratory device. In particular, the present invention relates to benchtop laboratory devices, comprising, but not limited to, incubators, centrifuges, and biosafety cabinets.

### BACKGROUND OF THE INVENTION

Such laboratory devices are usually used in laboratories and have different functionalities. In the case of some laboratory devices, for example, temperature control is also advantageous (e.g., with incubators or centrifuges). For example, to achieve such temperature control, laboratory devices can provide an exchange of air between the laboratory device and the environment (for example, the laboratory). It is possible in this case that, for example, particles from the laboratory device are released into the environment. A release of such particles (and in particular a large amount of particles) to the environment can be disadvantageous in some situations—in particular, it may be desirable, for example when working in clean rooms, that only a restricted amount of particles per unit of time are released from the laboratory device to the environment.

Current laboratory devices often cannot meet these requirements and release too large a quantity of particles per unit of time into the environment, which can be disadvantageous in many situations.

Embodiments of the present invention relate to overcoming or at least mitigating the disadvantages and deficiencies of the prior art. Accordingly, it is an object of the present invention to provide a laboratory device that releases relatively few particles into the environment or in which the release takes place in a relatively controlled manner.

### SUMMARY OF THE INVENTION

The present invention overcomes the foregoing and other shortcomings and drawbacks of laboratory devices with low particle emission heretofore known. While the invention will be discussed in connection with certain embodiments, it will be understood that the invention is not limited to these embodiments. On the contrary, the invention includes all alternatives, modifications, and equivalents as may be included within the spirit and scope of the invention.

According to one aspect, the present invention relates to a laboratory device which has an outer housing which defines an interior of the device. The laboratory device is designed to assume an operating state in which the pressure in the interior of the device is lower than the ambient pressure in the environment surrounding the laboratory device.

In particular, the laboratory device can be designed to generate a predetermined differential pressure in relation to an atmosphere surrounding the laboratory device, at least in part of the interior of the device. The outer housing has the advantage that particles which are generated in the interior of the device and/or are released into the air by the materials within the laboratory device are not released into the laboratory atmosphere in an uncontrolled manner, but are initially kept inside the laboratory device. With a lower pressure inside the outer housing, an uncontrolled outflow of air from the outer housing can be reduced, so that most or all of the particles contained in the air remain inside the outer housing. The air containing particles inside the interior of the device can be freed from particles by means of a filter apparatus, or at least the number of particles can be reduced.

Particles within the meaning of the present invention comprise small, delimited objects which can be detached in particular from solids and can be carried by a gaseous fluid, in particular air. Particles can also comprise aerosols, dust, fine dust, volatile organic compounds (VOCs), nanoparticles, and/or ultrafine particles.

The laboratory device advantageously achieves reduced particle emission, so that it is possible to operate the laboratory device within a clean room without further filter measures. In particular, the laboratory device can be operated in a clean room of ISO class 5 or better.

The corresponding laboratory device is therefore particularly suitable for use in a clean room.

In this regard, it should be understood that it is advantageous for laboratory devices to be operated in a clean region if contamination of the air, equipment surfaces, and/or laboratory surfaces can be controlled. For example, there are ISO standards in this regard, but also national or international guidelines, such as the EU guideline GMP Annex 1.

In particular, embodiments of the present invention make it possible to represent process and product quality in a reproducible manner by allowing the laboratory device to be used in clean rooms under controlled conditions, in order to reduce the risk of contamination, for example.

Embodiments of the present invention make it possible to keep the particle emission or the number of particles in the room in which the device is used as constant or as low as possible, which is particularly advantageous when the device is used in a technical clean room. Depending on the field of application, limit values for the particle emission of a device can be defined in terms of particle number and/or particle size. For technical clean rooms, reference can be made in particular to the ISO standard 14644 guideline.

Accordingly, devices brought into corresponding clean rooms should have defined particle emissions that do not exceed the defined particle emission limit values. This functionality can be provided by embodiments of the present invention.

Depending on their function, laboratory devices can exhibit a specific amount of exchange of air with the laboratory atmosphere. Laboratory devices in particular, which for example have a temperature control, pressure regulation function, and/or evaporation function, can have an exchange with the laboratory atmosphere, so that particles from the device can get into the laboratory atmosphere. The exchange of air can be necessary in the laboratory device, for example, for cooling, for heating, for evacuation, and/or for ventilation. Accordingly, the present invention can be applied in particular to such devices.

The greater the temperature difference between the laboratory device and the ambient temperature, the higher the particle emission from the laboratory device. Furthermore, laboratory devices with moving parts can have increased particle emission depending on the speed of parts of the laboratory device. Accordingly, it is advantageous, in particular for ovens, sterilizers, incubators, mixers, and centrifuges, to determine the particle emission and to reduce it according to the clean room requirements.

In embodiments of the present invention, it is possible for the surfaces in contact with the laboratory atmosphere to have as few emissions as possible in terms of particles and be compatible with standardized clean room cleaning methods.

Furthermore, in embodiments of the present invention, during an exchange of air between a volume of air in the laboratory device and the laboratory atmosphere, the particle input from the laboratory atmosphere into the volume of air in the laboratory device can be reduced. This can, for example, reduce the risk of sample contamination within the laboratory device. In particular, embodiments of the present invention aim to keep the atmosphere in the environment of the laboratory device as clean as possible by controlling the particle emission from the device. Opening the device in such an environment (e.g., to introduce samples into the device) is also beneficial for the cleanliness within the device, since the clean air in the environment (to which the device itself contributes) lowers the risk of contamination of the interior of the device.

Airborne particles can be harmful to laboratory samples, such as cells, and hazardous to laboratory personnel. Samples or products contaminated with particles can be defective, and a risk of contamination can increase with particle concentration. In particular in the case of thermally operated devices, particles can be released into the laboratory atmosphere in an uncontrolled manner via air currents. Such disadvantages can be avoided with embodiments of the present invention.

Overall, it should be understood that embodiments of the present invention relate to the reduction of particle emission, for example within specified ranges.

Overall, improved laboratory devices can thus be provided by means of embodiments of the present invention, which can also be suitable in particular for use in clean rooms.

The laboratory device can generate a difference between the ambient pressure and the pressure in the interior of the device, the pressure difference being in a range of from 1 Pa to 1000 Pa, preferably in the range of from 2 Pa to 500 Pa, more preferably in the range of from 5 Pa to 400 Pa.

The laboratory device can have an apparatus for generating the pressure difference. The apparatus for generating a pressure difference can be designed to continuously obtain a pressure difference. This can achieve the advantage that, independently of an absolute atmospheric pressure of the atmosphere surrounding the laboratory device, a relative, in particular constant, pressure difference can always be generated in the laboratory device. The apparatus for generating the pressure difference preferably generates a negative pressure so that air can penetrate into the laboratory device through unsealed regions of the laboratory device, but the escape of air from unsealed regions of the laboratory device can be prevented or at least reduced. In particular, air escaping from the laboratory device can be concentrated and/or restricted to an outlet of the apparatus for generating the pressure difference.

The apparatus for generating the pressure difference can comprise a fan and/or a pump. The fan achieves the advantage that a flow of air can be generated, which flow of air transports air within the interior of the device in the direction of the fan and then transports it out of the interior.

The apparatus for generating the pressure difference can be designed to achieve a conveying capacity in the range of from 10 to 40 times the total volume of the laboratory device per hour, preferably in the range of from 15 to 30 times the total volume of the laboratory device per hour. Depending on the conveying capacity, a quantity of particles can be transported from the interior of the device to the apparatus for generating the pressure difference. With a reduction in the conveying capacity, the quantity of particles that are transported can be controlled accordingly. This can achieve the advantage that air which leaves the laboratory device via an outlet of the apparatus for generating the pressure difference does not exceed a predetermined particle limit value, in particular a particle concentration limit value.

The laboratory device can comprise a controller which is designed to reduce a conveying capacity of the apparatus for generating the pressure difference, preferably to 1% to 20%, more preferably to 2% to 10%, for example to 2% to 5% of the overall conveying capacity. In particular, the controller can detect a physical parameter of the laboratory device, for example a temperature, and/or an operating mode of the laboratory device, and reduce a conveying capacity based on the physical parameter and/or the operating mode. As the device temperature rises, the emission of particles in the interior of the device can increase. For example, it is possible for the laboratory device to be operated with the reduced conveying capacity in a first operating state (for example in a normal operating mode) and to be operated with the full conveying capacity in a second operating state (for example in a cleaning mode in which the temperature is significantly increased). Thus, by controlling the conveying capacity, particle emission from the laboratory device can be limited, reduced, or at least kept below a predetermined limit value.

The apparatus for generating the pressure difference can have a maximum conveying capacity which corresponds to 20 to 30 air changes within one hour. Accordingly, the apparatus for generating the pressure difference can be designed to deliver a volume of air within one hour which corresponds to 20 to 30 times the air volume contained in the interior of the device. Alternatively, the maximum conveying capacity can be defined based on the total device volume of the laboratory device.

The apparatus for generating the pressure difference can be designed to convey gas from the interior of the device into the environment. In particular, the apparatus for generating the pressure difference can be designed, after an initial generation of a negative pressure, to transport an amount of air from the interior of the device, which amount corresponds to an inflowing air volume through unsealed regions of the laboratory device or is greater than this inflowing air volume. As a result, the negative pressure can be continuously maintained in the interior of the device.

The laboratory device can have a filter which is arranged between an outlet of the apparatus for generating the pressure difference and the environment of the laboratory device. The filter can in particular be arranged in the laboratory device in such a way that a volume of air, which is expelled by the apparatus for generating the pressure difference, is at least partially transported through the filter from the interior of the device into the laboratory atmosphere. Preferably, this discharge volume is passed entirely through the filter. The filter can be designed to filter particles from the air flowing through the filter in order to reduce a particle concentration in the air which is emitted by the laboratory device into the laboratory atmosphere. In particular, a filter can be used which is designed to reduce a maximum particle emission rate of the laboratory device below a predetermined limit value for use of the device in a clean room. Advantageously, the filter achieves particle filtration, which allows the laboratory device to be used in a clean room of ISO class 5 or better.

The filter can be a HEPA filter. The HEPA filter can be designed to separate suspended matter depending on an aerodynamic diameter of the suspended matter or particles. Particles that follow the flow of air around filter fibers of the filter can become attached to them as the particles approach the filter fibers. Furthermore, particles that cannot follow the flow of air around the filter fibers due to their size can collide with them and stick to them as a result of an impact. Particles with an aerodynamic diameter of less than 1 μm cannot follow the flow of air and, for example, collide with the filter fibers due to random movement and stick to them.

The filter can have a separation efficiency of at least 99%, preferably at least 99.9%, more preferably at least 99.95%, even more preferably at least 99.995%, based on particles with a particle size which are most difficult to separate. These particles are usually particles with a particle size in the range of from about 0.1 μm to about 0.3 μm.

The filter can be made of a fibrous material, for example glass fiber.

The filter can be releasably attached. In particular, the filter can be releasably attached to the outer housing. In this way, the advantage can be achieved that the filter can be replaced when it becomes saturated with particles. For example, single-use filters can be used.

The filter can comprise a replaceable filter material, in which case the filter material in the filter can be replaced, cleaned, or reactivated in order to restore an original filter performance.

The laboratory device can comprise a protective screen which is arranged on the filter so that the filter can be protected from external damage. The protective screen can have regular, in particular hexagonal, openings. The protective screen may form part of the outer housing, and/or the protective screen may be arranged to be set back from a housing surface of the outer housing.

The outer housing may include a side housing, a rear housing, a ceiling housing, a lower housing, and a door housing. The rear housing and the door housing can be arranged at opposite ends of the laboratory device. The individual housing parts can be connected to one another in a sealed manner in order to reduce or prevent particle emission at joints and/or connection points. A negative pressure can be generated at the remaining air inlets of the outer housing by means of the apparatus for generating the negative pressure in relation to the ambient pressure, so that air can flow from the environment into the interior of the device at the remaining air inlets in order to reduce particle emission.

The filter may be arranged in the rear housing. As a result, the advantage can be achieved that the exhaust air, which is released from the laboratory device into the laboratory atmosphere, exits from the laboratory device at a distance from the door opening of the laboratory device. Accordingly, a probability that samples processed in the laboratory device are exposed to particles emitted by the laboratory device can be reduced. Accordingly, the risk of contamination can be reduced. Furthermore, the exhaust air exiting at the rear can be efficiently fed to a further exhaust air treatment, for example particle separation and/or air extraction. In particular, the filter can be aligned perpendicularly to an installation level of the laboratory device. As a result, a depth of the rear housing part can be reduced in order to minimize the overall depth of the laboratory device.

The laboratory device can comprise a filter holder on which the filter is arranged, wherein the filter holder can form part of the outer housing and/or the filter holder can be releasably connected to the outer housing. The filter holder can in particular be screwed to the rear housing.

The filter may be arranged on an inside or an outside of the outer housing.

The laboratory device may have a total volume in the range of from 0.1 m³ to 2.5 m³, preferably in the range of from 0.2 m³ to 1.0 m³, and more preferably in the range of from 0.4 m³ to 0.8 m³. This allows the laboratory device to be positioned on or under laboratory tables. Furthermore, the laboratory device can be movably arranged in a laboratory.

The laboratory device can be an incubator.

The laboratory device can be a centrifuge.

The laboratory device can have an incubation chamber. The function of the incubation chamber can be independent of the function of the apparatus for generating a pressure difference in the interior of the device. The incubation chamber can have a door which can form a separate chamber door independent of the door housing of the laboratory device. In a closed state, in which the chamber door and/or the door housing are closed, the incubation chamber is advantageously separated from the interior of the device in a fluid-tight, gas-tight, and/or particle-tight manner.

The laboratory device can have a carbon dioxide (CO₂) sensor which is designed to determine the CO₂ content of the surrounding atmosphere independently of a sensor temperature, humidity of the surrounding atmosphere, the oxygen content of the surrounding atmosphere, and a barometric pressure of the surrounding atmosphere. The CO₂ sensor can be a MEMS sensor. Furthermore, the CO₂ sensor can have a recovery time of less than 5 minutes. A recovery time can be defined as a period of time until predetermined incubation chamber conditions are reached after opening the chamber door. The CO₂ sensor can be arranged in the incubation chamber.

The laboratory device can have an apparatus for adjusting the oxygen content in the incubation chamber. The apparatus for adjusting the oxygen content can be designed to regulate the oxygen content to simulate hypoxic conditions in a range of from 1% to 21% oxygen. This oxygen level can be beneficial in particular for primary cells and applications in stem cell and embryo research.

The apparatus for adjusting the oxygen content can be designed to regulate the oxygen content to simulate hyperoxic conditions in a range of from 5% to 90% oxygen. These conditions can be beneficial for lung tissue, retinal tissue, or other delicate tissue types.

The laboratory device can have an apparatus for controlling the humidity within the incubation chamber. In general, the laboratory device can be humidified passively or actively. For example, it can be an actively humidified incubator. For example, the apparatus for controlling the humidity may comprise an evaporator oven or an aerosol generator. Alternatively, the laboratory device can be humidified passively. In such a case, for example, the humidity within the incubation chamber can be increased by evaporating water in an evaporating dish. In general, the incubation chamber can have a cold spot which has a reduced temperature relative to the other inner surfaces of the incubation chamber, so that moisture condenses at the cold spot. Air humidity within the incubation chamber can be reduced by means of the cold spot.

The apparatus for generating the pressure difference and the filter can form a particle emission control system which is designed to limit the number of particles emitted by the laboratory device. In particular, the laboratory device can have a controller which is designed to detect the condition of the filter and to adapt a conveying capacity of the apparatus for generating the pressure difference to the state of the filter. In particular, with increasing particle emission within the interior of the device, saturation of the filter with particles can be prevented by reducing the conveying capacity.

The laboratory device can have a heating apparatus which is designed to at least partially control the temperature of the laboratory device. The heating apparatus can be designed to control the temperature of an incubation chamber for incubating samples and/or to control the temperature of the incubation chamber for sterilization. Preferably, the incubation chamber reaches a temperature of 180° C. during the sterilization, in particular all inner surfaces of the incubation chamber reach a temperature of at least 180° C. for a predetermined time.

The laboratory device may have an inner housing that defines a chamber. The chamber may be arranged in the outer housing. Advantageously, outer walls of the chamber may be arranged at a distance from inner surfaces of the outer housing. The inner housing can accordingly be thermally insulated from the laboratory atmosphere. In the case of an incubator, the chamber can in particular be the incubation chamber.

The inner housing may have side walls, a back wall, a lower wall, a ceiling wall, and a door portion. The respective portions can be aligned with the corresponding portions of the outer housing. In particular, the door portion may be arranged parallel and adjacent to the door housing of the outer housing such that the chamber is accessible through the door portion and the door housing.

At least parts of the inner housing can be made of metal.

The metal of the inner housing can be copper or steel, preferably electropolished stainless steel.

The side walls, the back wall, the lower wall, and/or the ceiling wall can be made of metal.

At least parts of the outer housing (10) can be made of metal.

The metal can be steel.

The metal may be stainless and/or brushed steel, and preferably brushed 304 stainless steel. Metal and in particular stainless steel can realize the advantage of providing a reduced particle emission, in particular at an increased relative temperature of the laboratory device in relation to a temperature of the laboratory atmosphere. Furthermore, metals can have improved compatibility with standardized clean room cleaning methods, so that particle deposits on the surfaces can be efficiently removed. Furthermore, at least a selection of surfaces of the laboratory device can be polished, in particular electropolished. Polishing can reduce deposition of particles on the surface, reduce emission of particles from the surface, and/or facilitate removal of particles from the surface.

In the operating state, the pressure in the interior of the device can be in a range delimited outside by the outer housing and inside by the inner housing, and this pressure can be lower than a pressure in the chamber. Advantageously, although the chamber is arranged in the laboratory device, it is not part of the interior of the device, which is fluidically connected to the apparatus for generating the pressure difference. Typically, ambient pressure may prevail in the chamber. In particular, the laboratory device can be equipped with an apparatus that adjusts the pressure in the chamber to the ambient pressure.

The laboratory device can comprise a switch box, in which case the apparatus for generating pressure can be designed to generate a negative pressure in the switch box. The switch box is advantageously formed in the rear region. The apparatus for generating the pressure difference can be arranged in the switch box. The switch box can be separated from the rest of the interior of the device by a wall, wherein the wall has a wall surface and at least one opening. The corresponding regions can therefore be fluidly connected to one another by means of the at least one opening.

The apparatus for generating the differential pressure can be arranged in a rear region which is delimited by the rear wall and the rear housing. Side regions of the interior of the device can be delimited by the side walls and the side housing.

The at least one opening can have a total cross-sectional area (i.e., the sum of the cross-sectional areas of the one or more openings) which is in the range of from 0.1% to 20% of the wall region, preferably in the range of from 0.5% to 10% of the wall region, more preferably in the range of from 1% to 5% of the wall region. The at least one opening can generate a predetermined volume flow from the side housing to the rear housing depending on a conveying capacity of the apparatus for generating the differential pressure. As a result, the particle input from the side region into the rear region and finally into the filter can also be predetermined. The opening can be formed by a plurality of passages. In particular, the opening can comprise a plurality of through holes.

The laboratory device can be designed to generate a negative pressure in a door region that is delimited by the door housing and the door portion. The door housing can be fluidically coupled to the side regions and/or to the rear region in order to generate a flow of a volume of air from the door housing to the apparatus for generating the pressure difference.

The laboratory device can comprise at least one hose that fluidly connects the apparatus for generating the differential pressure to at least one other region.

The at least one hose can fluidly connect the door region and the apparatus for generating the differential pressure. For example, the hose can be connected to the pump. A hose end can be arranged at a position of the door housing which can have an increased particle emission, so that the particles can be transported through the hose to the apparatus for generating the differential pressure.

The at least one hose may fluidly connect the apparatus for generating the pressure difference to a front region which is delimited by the side housing, the side walls, the ceiling housing, the ceiling wall, the lower housing, and the lower wall, and is adjacent to the door portion.

The at least one hose can comprise at least one branch, to which at least two hose segments are connected, each having a hose opening.

The hose openings of the hose segments can be arranged at a distance from one another.

The hose openings can be arranged in the door region. Accordingly, a plurality of locations within the door region can be reached by means of the hose for suction of particles. For example, a hose can be perforated in order to drain particles from the environment of the hose. A single hose segment can be arranged in the door housing for this purpose.

The hose can be arranged at least partially in a hollow connecting element, the hollow connecting element being designed to realize a connecting channel between the door housing and the rear housing and/or the side housing and/or the ceiling housing.

The at least one hose can be made of a material that has a temperature resistance of up to at least 200° C., preferably up to at least 220° C.

The at least one hose can be made of silicone.

The laboratory device can have a flow channel which connects the apparatus for generating the pressure difference to an outlet opening in the outer housing. This can achieve the advantage that the apparatus for generating the pressure difference can be arranged spatially separately from the outlet opening. Furthermore, components that require a flow of cooling air, in particular electronic components, can be arranged in the flow channel. A part of the flow channel can be formed by the switch box. The flow channel can connect the inner housing and the outer housing or be delimited by the inner housing and the outer housing.

The filter can be arranged in the flow channel and/or at the outlet opening. Accordingly, the advantage can be achieved that the volume of air, which flows through the flow channel by means of the apparatus for generating the pressure difference, has a reduced number of particles when it exits the laboratory device.

The apparatus for generating the pressure difference can be arranged on the flow channel. In particular, an outlet of a pump of the apparatus for generating the pressure difference can be arranged in the flow channel.

The apparatus for generating the pressure difference can have an outlet opening which is connected to the flow channel. In this way, air can be sucked into the interior of the device, and air can be blown out of the laboratory device into the laboratory atmosphere through the outlet portion.

The filter can close off the outer portion of the flow channel such that a volume of air flowing through the flow channel completely passes through the filter.

The laboratory device can comprise at least one thermal insulation component which consists at least partially of a thermal insulation material. The thermal insulation material is advantageously flexible and/or compressible; in particular, glass wool or mineral wool can be used. As a result, the advantage can be achieved that existing cavities have a high filling density with insulation material, so that the device (for example an incubation chamber in the device) is suitably insulated from the outside atmosphere. The insulation material can have a temperature resistance of at least 200° C., preferably at least 220° C.

The at least one thermal insulation component can consist at least partially of a thermal insulation material.

The at least one thermal insulation component can be arranged in an intermediate space between the outer housing and the inner housing. In this way, thermal insulation in particular can be improved, so that greater temperature stability can be achieved within the chamber. Advantageously, a flat insulation component is provided on all sides of the inner housing.

The at least one thermal insulation component can be arranged between the side walls and the side housing, between the rear wall and the rear housing, between the lower wall and the lower housing, between the ceiling wall and the ceiling housing, and/or between the door portion and the door housing.

The at least one thermal insulation component can have a final layer which seals the at least one thermal insulation component. As a result, the advantage can be achieved that particle emission from the thermal insulation material is reduced. In particular, an increase in particle emission as the device temperature increases can be reduced, so that not only are fewer particles released overall from the thermal insulation component, but an increase in particle emission as the temperature rises can also be slowed down. The final layer advantageously completely encloses the thermal insulation material.

The final layer can comprise a film. The film can in particular form a particle emission protection layer which abuts the insulation material and/or is bonded to it. The film can form a closed volume in which the thermal insulation material is arranged in order to shield the thermal insulation material from the atmosphere in the interior of the device or from the laboratory atmosphere.

The film can have a temperature resistance of up to at least 200° C., preferably up to at least 220° C. More preferably, the temperature resistance of the film can be in the range of from 200° C. to 300° C. As a result, the advantage can be achieved that during an operating mode of the laboratory device with a correspondingly high device temperature, the film continues to have an emission-reducing effect. In particular, an emission of particles from the film itself can be reduced. Advantageously, the film has a temperature-independent particle emission rate, or the particle emission rate is at least only slightly dependent on a temperature of the film.

The film can be designed to be flexible and/or low-emission. In this way, the advantage of efficient processing can be achieved when joining the thermal insulation material and the film together. Furthermore, the film is exposed to the device atmosphere and the corresponding differential pressure, so that particle emission from the film is also part of the total particle emission from the laboratory device. The lower the particle emission of the film itself, the lower the total particle emission of the laboratory device. In particular, the film can be impermeable or emission-reducing for particles of the thermal insulation material.

The film can comprise an adhesive layer which is designed to bond overlapping layers of the film in order to seal the thermal insulation component, in particular to seal it in a particle-tight manner or to reduce particle emission. As a result, the advantage can be achieved that a consistently high reduction in particle emission can be achieved in the overlapping regions of the film. The adhesive layer can also have a temperature stability of at least 200° C., preferably at least 220° C.

The adhesive layer can be designed to bond the final layer to the thermal insulation material. The adhesive layer can be designed to be permanently flexible in order to follow the thermally induced expansion of the film. With the connection of the adhesive layer to a surface of the thermal insulation material, a particle emission of the thermal insulation material can be reduced at the corresponding surface. The adhesive layer can have the function of a particle filter or a particle trap. This effect can be reduced with saturation of the adhesive layer with particles.

The thermal insulation material can be wrapped in the final layer. Furthermore, overlapping regions of the film can be bonded to cover another film portion. As a result, the advantage can be achieved that a particle emission reduction similar to the non-adhered regions of the film can be brought about at cut edges. In particular, imperfections that occur in the first adhesion of the film can be compensated for by the adhesion of the covering film portion.

The film can be a plastics film which preferably contains a polymer.

The film can comprise polyamide. Thereby, the advantage can be achieved that the final layer is waterproof, dimensionally stable, tear-resistant, highly elastic, and durable.

The final layer can comprise a film which is bonded by means of a polyamide tape. The polyamide tape can have a silicone adhesive layer which is designed to bond film portions of the final layer and/or to connect the final layer to the thermal insulation component.

The adhesive layer can be a silicone adhesive layer. The silicone adhesive layer can form one layer of the film. In particular, the film can have a two-layer structure, a first layer being a polyamide layer and a second layer being a silicone layer.

The laboratory device can comprise a control unit which is designed to adapt a conveying capacity of the apparatus for generating the pressure difference to an operating mode of the laboratory device. In this way, the advantage can be achieved in particular that the conveying capacity can be adjusted to a particle emission rate in the interior of the device, or through the components of the laboratory device.

The control unit can have a first operating mode and the apparatus for generating the differential pressure can comprise a fan and a pump, with only the fan being active in the first operating mode.

The control unit can have a second operating mode, with both the fan and the pump being active in the second operating mode. As a result, the advantage can be achieved that the pump can be used to guide particles in portions of the interior of the device to the filter, which particles are exposed to an insufficiently low flow of air due to the fan alone. Efficient particle transport to the apparatus for generating the pressure difference and/or to the filter can advantageously be realized by means of a hose system which has hose openings which are arranged in regions with increased particle emission. In particular, with increasing distance from the apparatus for generating the pressure difference, housing portions can have an insufficient flow of air in the direction of the apparatus for generating the pressure difference, so that a transport volume of particles from this region can be reduced.

At least one hose opening is advantageously arranged in the door region in order to remove particles from the door region. In particular, the door region can be fluidically coupled to the apparatus for generating the pressure difference by means of the hose.

The control unit can be designed to switch from the first operating mode to the second operating mode when a temperature limit value is reached. The device temperature can be a measure of a particle emission rate of the laboratory device. Accordingly, the advantage can be achieved that a conveying capacity of the apparatus for generating the pressure difference scales with the particle emission rate of the laboratory device. In particular, housing parts, which have a particularly temperature-dependent particle emission rate or which experience a low volume flow in the first operating mode, can be connected to the apparatus for generating the pressure difference so as to be more efficient in terms of flow in the second operating mode.

The conveying capacity can be adjusted depending on a temperature of the laboratory device or a part of the laboratory device. When using a pump and a fan, a conveying capacity can be selectively adjusted in each case in order to ensure adequate particle transport from the housing regions which are mainly reached by the fan, and from regions which are mainly reached by the pump. Furthermore, the conveying capacity can be increased when particle emission increases, for example in order not to exceed a specified emission value.

A conveying capacity of the fan can be constant and a conveying capacity of the pump can be adjustable in order to increase an overall conveying capacity of the apparatus for generating the pressure difference.

The pump can be switched on in addition to the fan when a predetermined temperature of the laboratory device is reached in order to increase the conveying capacity.

The apparatus for generating the pressure difference can be designed to suck particles out of the interior of the outer housing, in particular the door region.

The apparatus for generating pressure can have a minimum conveying capacity in order to generate a directed flow of air from the environment of the laboratory device into the outer housing. This can ensure that a negative pressure is always generated in the interior of the device. Furthermore, the particle emission of the laboratory device can be minimal at a minimal conveying rate.

The switch box may comprise a switch box component. The apparatus for generating the differential pressure can be designed to generate a flow of air in the switch box and to control the flow of air depending on a component temperature of the switch box component. As a result, components in the switch box can be cooled by the flow of air generated by the apparatus for generating the differential pressure.

The switch box component can be a cooling element, in particular a cooling bracket.

The cooling element can be arranged at least partially on the inner housing. Advantageously, the cooling element is thermally coupled to a housing wall of the inner housing to create a reduced temperature surface on an inner wall of the inner housing. Moisture can condense on this surface.

The laboratory device can comprise a closing apparatus which is designed to connect the door housing to another part of the outer housing in a closing manner. The closing apparatus can be air-filled. In particular, the air-filled closing apparatus can be arranged on a stop of the door housing on the side housing or ceiling housing, preferably on the lower housing. Furthermore, a hose opening of the apparatus for generating the differential pressure can be arranged on or in the air-filled closing apparatus.

The laboratory device can be designed to generate a negative pressure in the closing apparatus. Correspondingly, an emission of particles from the air-filled closing apparatus can be reduced.

The outer housing can be at least partially airtight. In particular, the outer housing can be sealed in such a way that existing housing openings are arranged in an effective region of the apparatus for generating the differential pressure in order to prevent or at least reduce the escape of particles in these regions.

The thermal insulation components can be arranged in the interior of the device such that air ducts are formed between the thermal insulation components and an inner wall of the outer housing and/or between the thermal insulation components and an outer wall of the inner housing. In particular, there can be a network of air ducts, with the apparatus for generating the differential pressure being designed to generate a negative pressure or a flow of air in these air ducts in order to transport particles in the direction of the apparatus for generating the differential pressure. The air ducts can form a flow cross section in the corresponding housing portion, with the sum of the air ducts that can be reached through the apparatus for generating the differential pressure forming a total flow cross section over which the conveying capacity of the apparatus for generating the differential pressure is distributed. In addition, hoses connected to the pump can form part of the total flow cross section.

The air ducts can be part of the flow channel.

The present invention also relates to the use of the described laboratory device in a clean room.

The use can be for the incubation of organisms, cells, bacteria, and/or viruses.

The use can be for the sterilization of an inner housing of the laboratory device.

The present invention is also further described with reference to the following numbered embodiments.

Device embodiments are named below. These embodiments are abbreviated with the letter “D” followed by a number. Whenever reference is made below to “device embodiment,” these embodiments are intended.

D1. Laboratory device (1), the laboratory device (1) having an outer housing (10) which defines an interior of the device, wherein the laboratory device (1) is designed to assume an operating state at which a pressure in the interior of the device is lower than an ambient pressure in the environment of the laboratory device (1).

D2. Laboratory device (1) according to the preceding embodiment, wherein a difference between the ambient pressure and the pressure is in the range of from 1 Pa to 1000 Pa, preferably in the range of from 2 Pa to 500 Pa, more preferably in the range of from 5 Pa to 400 Pa.

D3. Laboratory device (1) according to any of the preceding embodiments, wherein the laboratory device (1) has an apparatus for generating the pressure difference.

D4. Laboratory device (1) according to the preceding embodiment, wherein the apparatus for generating the pressure difference comprises a fan (30) and/or a pump (32).

D5. Laboratory device (1) according to any of the preceding embodiments having the features of embodiment D3, wherein the apparatus for generating the pressure difference is designed to achieve a conveying capacity in the range of from 10 times to 40 times the total volume of the laboratory device per hour, preferably in the range of from 15 to 30 times the total volume of the laboratory device per hour.

D6. Laboratory device (1) according to any of the preceding embodiments having the features of embodiment D3, wherein the laboratory device (1) comprises a controller which is designed to reduce a conveying capacity of the apparatus for generating the pressure difference, preferably to 1% to 20%, more preferably to 2% to 10%, for example to 2% to 5% of an overall conveying capacity of the apparatus for generating the pressure difference.

D7. Laboratory device (1) according to any of the preceding embodiments having the features of embodiment D3, wherein the apparatus for generating the pressure difference is designed to convey gas from the interior of the device into the environment of the laboratory device (1).

D8. Laboratory device (1) according to the preceding embodiment, wherein the laboratory device (1) has a filter (50) which is arranged between an outlet of the apparatus for generating the pressure difference and the environment of the laboratory device (1).

D9. Laboratory device (1) according to the preceding embodiment, wherein the filter (50) is a HEPA filter.

D10. Laboratory device (1) according to any of the preceding embodiments having the features of embodiment D8, wherein the filter (50) is releasably attached.

D11. Laboratory device (1) according to any of the preceding embodiments having the features of embodiment D8, wherein the filter (50) comprises a replaceable filter material.

D12. Laboratory device (1) according to any of the preceding embodiments having the features of embodiment D8, wherein the laboratory device (1) comprises a protective screen (39) which is arranged on the filter (50).

D13. Laboratory device (1) according to any of the preceding embodiments, wherein the outer housing (10) has

a side housing (12),

a rear housing (18),

a ceiling housing (14),

a lower housing (16), and

a door housing (19),

wherein the rear housing (18) and the door housing (19) are arranged at opposite ends of the laboratory device (1).

D14. Laboratory device (1) according to the preceding embodiment having the features of embodiment D8, wherein the filter (50) is arranged in the rear housing (18).

D15. Laboratory device (1) according to any of the preceding embodiments having the features of embodiment D13, wherein the laboratory device (1) comprises a filter holder (37) which forms part of the outer housing (10) and/or wherein the filter holder (37) is releasably connected to the outer housing (10).

D16. Laboratory device (1) according to any of the preceding embodiments having the features of embodiments D8 and D13, wherein the filter (50) is arranged on an inside or an outside of the outer housing (10).

D17. Laboratory device (1) according to any of the preceding embodiments, wherein the laboratory device has a total volume in the range of from 0.1 m³ to 2.5 m³, preferably in the range of from 0.2 m³ to 1.0 m³, and more preferably in the range of from 0.4 m³ to 0.8 m³.

D18. Laboratory device (1) according to any of the preceding embodiments, wherein the laboratory device (1) is an incubator.

D19. Laboratory device (1) according to any of the embodiments D1 to D17, wherein the laboratory device (1) is a centrifuge.

D20. Laboratory device (1) according to any of the preceding embodiments having the features of embodiment D18, wherein the laboratory device (1) has an incubation chamber.

D21. Laboratory device (1) according to any of the preceding embodiments having features of embodiments D3 and D8, wherein the apparatus for generating the pressure difference and the filter (50) form a particle emission control system which is designed to count the number of particles emitted by the laboratory device (1).

D22. Laboratory device (1) according to any of the preceding embodiments, wherein the laboratory device (1) has a heating apparatus.

D23. Laboratory device (1) according to any of the preceding embodiments, wherein the laboratory device (1) has an inner housing (20) which defines a chamber.

D24. Laboratory device (1) according to the preceding embodiment, wherein the inner housing (20) has

side walls (22),

a back wall (28),

a lower wall (26),

a ceiling wall (24), and

a door portion (29).

D25. Laboratory device (1) according to any of the 2 preceding embodiments, wherein at least parts of the inner housing (20) are made of metal.

D26. Laboratory device (1) according to any of the preceding embodiments having the features of embodiment D22, comprising an apparatus for controlling the humidity in the chamber.

D27. Laboratory device (1) according to any of the preceding embodiments having the features of embodiment D25, wherein the metal of the inner housing (20) is copper or steel, preferably electropolished stainless steel.

D28. Laboratory device (1) according to any of the preceding embodiments having the features of embodiments D24 and D25, wherein the side walls (22), the rear wall (28), the lower wall (26) and the ceiling wall (24) are made of metal.

D29. Laboratory device (1) according to any of the preceding embodiments, wherein at least parts of the outer housing (10) are made of metal.

D30. Laboratory device (1) according to the preceding embodiment, wherein the metal is steel.

D31. Laboratory device (1) according to the preceding embodiment, wherein the metal is stainless and/or brushed steel, and preferably brushed 304 stainless steel.

D32. Laboratory device (1) according to any of the preceding embodiments having the features of embodiments D3 and D23, wherein, in the operating state, the pressure in the interior of the device is present in a region that is delimited outside by the outer housing (10) and inside by the inner housing (20), and wherein this pressure is less than a pressure in the chamber.

D33. Laboratory device (1) according to any of the preceding embodiments having the features of embodiment D3, wherein the laboratory device (1) comprises a switch box, wherein the apparatus for generating the differential pressure is designed to generate a negative pressure in the switch box, wherein the switch box is separated from the rest of the interior of the device by a wall, wherein the wall has a wall surface, and wherein the wall has at least one opening.

D34. Laboratory device (1) according to any of the preceding embodiments having the features of embodiments D3, D13, and D24, wherein the apparatus for generating the differential pressure is arranged in a rear region which is delimited by the rear wall (28) and the rear housing (18), and wherein side regions of the interior of the device are delimited by the side walls (22) and the side housings (12).

D35. Laboratory device (1) according to any of the preceding embodiments having the features of embodiment D33, wherein the at least one opening has a total cross-sectional area in the range of from 0.1% to 20% of the wall region, preferably in the range of from 0.5% to 10% of the wall region, more preferably in the range of from 1% to 5% of the wall region, and wherein the at least one opening preferably has a predetermined volume flow from the side housing (12) to the rear housing (18) generated depending on a conveying capacity of the apparatus for generating the differential pressure.

D36. Laboratory device (1) according to any of the preceding embodiments having the features of embodiments D3, D13, and D24, wherein the laboratory device (1) is designed to generate a negative pressure in a door region which is delimited by the door housing (19) and the door portion (29).

D37. Laboratory device (1) according to any of the preceding embodiments having the features of embodiment D3, wherein the laboratory device (1) comprises at least one hose which fluidly connects the apparatus for generating the differential pressure to at least one other region.

D38. Laboratory device (1) according to the preceding embodiment and having the features of embodiment D36, wherein the at least one hose fluidly connects the door region and the apparatus for generating the differential pressure.

D39. Laboratory device (1) according to any of the preceding embodiments having the features of embodiments D13, D24, and D37, wherein the at least one hose fluidly connects the apparatus for generating the pressure difference to a front region which is delimited by the side housing (12), the side walls (22), the ceiling housing (14), the ceiling wall (24), the lower housing (16), and the lower wall (26), and is adjacent to the door portion (29).

D40. Laboratory device (1) according to any of the preceding embodiments having the features of embodiment D37, wherein the at least one hose comprises at least one branch to which at least two hose segments are connected, each having at least one hose opening.

D41. Laboratory device (1) according to the preceding embodiment, wherein the hose openings of the at least two hose segments are arranged at a distance from one another.

D42. Laboratory device (1) according to the preceding embodiment and having the features of embodiment D36, wherein the hose openings are arranged in the door region.

D43. Laboratory device according to any of the preceding embodiments having the features of embodiment D37, wherein the at least one hose is made of a material that has a temperature resistance of up to at least 200° C., preferably up to at least 220° C.

D44. Laboratory device according to any of the preceding embodiments having the features of embodiment D37, wherein the at least one hose is made of silicone.

D45. Laboratory device (1) according to a previous embodiment having the features of embodiment D3, wherein the laboratory device (1) has a flow channel which connects the apparatus for generating pressure to an outlet opening in the outer housing (10).

D46. Laboratory device (1) according to any of the preceding embodiments having the features of embodiments D8 and D45, wherein the filter (50) is arranged in the flow channel and/or at the outlet opening.

D47. Laboratory device (1) according to any of the preceding embodiments having the features of embodiments D3 and D45, wherein the apparatus for generating the pressure difference is arranged on the flow channel.

D48. Laboratory device (1) according to any of the preceding embodiments having the features of embodiments D3 and D45, wherein the apparatus for generating the pressure difference has an outlet opening which is connected to the flow channel.

D49. Laboratory device (1) according to any of the preceding embodiments having the features of embodiments D8 and D45, wherein the filter (20) closes off the outer portion of the flow channel.

D50. Laboratory device (1) according to any of the preceding embodiments, comprising at least one thermal insulation component.

D51. Laboratory device (1) according to the preceding embodiment, wherein the at least one insulation component consists at least partially of a thermal insulation material.

D52. Laboratory device (1) according to the preceding embodiment and having the features of embodiment D23, wherein the at least one thermal insulation component is arranged in an intermediate space between the outer housing (10) and the inner housing (20).

D53. Laboratory device (1) according to the preceding embodiment having the features of embodiments D13 and D24, wherein the at least one thermal insulation component is arranged between the side walls (22) and the side housing (12), between the rear wall (28) and the rear housing (18), between the lower wall (26) and the lower housing (16), between the ceiling wall (24) and the ceiling housing (14), and/or between the door portion (29) and the door housing (19).

D54. Laboratory device (1) according to any of the preceding embodiments having the features of embodiment D51, wherein the at least one thermal insulation component has a final layer which seals the at least one thermal insulation component.

D55. Laboratory device (1) according to the preceding embodiment, wherein the final layer comprises a film.

D56. Laboratory device (1) according to the preceding embodiment, wherein the film has a temperature resistance of up to at least 200° C., preferably up to at least 220° C.

D57. Laboratory device (1) according to any of the preceding embodiments having the features of embodiment D5, wherein the film is flexible and/or low-emission.

D58. Laboratory device (1) according to any of the preceding embodiments having the features of embodiment D55, wherein the film comprises an adhesive layer which is designed to bond overlapping layers of the film to seal the thermal insulation component, in particular to seal it in a particle-tight or particle-emission-reducing manner.

D59. Laboratory device (1) according to the preceding embodiment and having the features of embodiment D51, wherein the adhesive layer is designed to bond the final layer to the thermal insulation material.

D60. Laboratory device (1) according to any of the preceding embodiments having the features of embodiment D49, wherein the thermal insulation material is wrapped in the film, and/or wherein overlapping regions of the film are bonded to cover another film portion.

D61. Laboratory device (1) according to any of the preceding embodiments having the features of embodiment D55, wherein the film is a plastics film which preferably contains a polymer.

D62. Laboratory device (1) according to the preceding embodiment, wherein the film comprises polyamide.

D63. Laboratory device (1) according to any of the preceding embodiments having the features of embodiment D61, wherein the final layer is bonded by means of a polyamide tape.

D64. Laboratory device (1) according to any of the preceding embodiments having the features of embodiment D58, wherein the adhesive layer is a silicone adhesive layer.

D65. Laboratory device (1) according to any of the preceding embodiments having the features of embodiment D3, comprising a control unit which is designed to adapt a conveying capacity of the apparatus for generating the pressure difference to an operating mode of the laboratory device (1).

D66. Laboratory device (1) according to the preceding embodiment, wherein the control unit has a first operating mode and the apparatus for generating the differential pressure comprises a fan (30) and a pump (32), wherein only the fan (30) is active in the first operating mode.

D67. Laboratory device (1) according to the preceding embodiment, wherein the control unit has a second operating mode, wherein both the fan (30) and the pump (32) are active in the second operating mode.

D68. Laboratory device (1) according to the preceding embodiment, wherein the control unit is designed to switch from the first operating mode to the second operating mode when a temperature limit value is reached.

D69. Laboratory device (1) according to any of the preceding embodiments having the features of embodiment D4 and D66, wherein a conveying capacity of the fan (30) is constant, and a conveying capacity of the pump (32) is adjustable in order to increase the overall conveying capacity of the apparatus for generating the pressure difference.

D70. Laboratory device (1) according to any of the preceding embodiments having the features of embodiment D4 and D66, wherein the control unit is designed to switch on the pump (32) in addition to the fan (30) when a predetermined temperature of the laboratory device (1) is reached, in order to increase a conveying capacity.

D71. Laboratory device (1) according to any of the preceding embodiments having the features of embodiment D3 and D36, wherein the apparatus for generating the pressure difference is designed to suck particles out of an interior of the outer housing (10), in particular the door region.

D72. Laboratory device (1) according to any of the preceding embodiments having the features of embodiment D3, wherein the apparatus for generating pressure has a minimum conveying capacity in order to generate a directed flow of air from the environment of the laboratory device (1) into the outer housing (10).

D73. Laboratory device (1) according to any of the preceding embodiments having the features of embodiment D33, wherein the switch box comprises a switch box component, and wherein the apparatus for generating pressure is designed to generate a flow of air in the switch box and to control the flow of air depending on a component temperature of the switch box component.

D74. Laboratory device (1) according to the preceding embodiment D67, wherein the switch box component is a cooling element, in particular a cooling bracket.

D75. Laboratory device (1) according to the preceding embodiment and having the features of embodiment D24, wherein the cooling element is at least partially arranged on the inner housing (20).

D76. Laboratory device (1) according to any of the preceding embodiments having the features of embodiment D13, comprising a closing apparatus (35) which is designed to connect the door housing (19) to another part of the outer housing (10) in a closing manner.

D77. Laboratory device (1) according to the preceding embodiment, wherein the laboratory device is designed to generate a negative pressure in the closing apparatus (35).

D78. Laboratory device (1) according to any of the preceding embodiments, wherein the outer housing (10) is at least partially airtight.

D79. Laboratory device (1) according to any of the preceding embodiments having the features of embodiment D8, wherein the filter has a degree of separation of at least 99%, preferably at least 99.9%, more preferably at least 99.95%, even more preferably at least 99.995%, based on particles with a particle size that is most difficult to separate.

D80. Laboratory device (1) according to any of the preceding embodiments having the features of embodiment D8, wherein the filter is made of a fiber material, for example glass fiber.

Use embodiments are mentioned below. These embodiments are abbreviated with the letter “U” followed by a number. Whenever reference is made herein to “use embodiments,” these embodiments are meant.

U1. Use of the laboratory device (1) according to any of the preceding embodiments in a clean room.

U2. Use of the laboratory device (1) according to any of the preceding device embodiments for the incubation of organisms, cells, bacteria, and/or viruses.

U3. Use of the laboratory device (1) according to any of the preceding device embodiments for the sterilization of an inner housing of the laboratory device.

Various additional features and advantages of the invention will become more apparent to those of ordinary skill in the art upon review of the following detailed description of the illustrative embodiments taken in conjunction with the accompanying drawings.

### BRIEF DESCRIPTION OF THE DRAWINGS

The present invention will now be described with reference to the accompanying drawings which illustrate embodiments of the present invention. These embodiments exemplify and do not limit the present invention.

FIG. 1 is a schematic vertical cross-sectional view of an embodiment of the laboratory device;

FIG. 2 is a schematic vertical cross-sectional view of an embodiment of the laboratory device;

FIG. 3 is a perspective rear view of the laboratory device;

FIG. 4 is an enlarged portion of FIG. 3, particularly showing a filter;

FIG. 5 is a perspective front view of the laboratory device with the door closed;

FIG. 6 is the perspective front view with the door open; and

FIG. 7 is a schematic horizontal cross-sectional view of the laboratory device.

### DETAILED DESCRIPTION OF THE INVENTION

It is noted that not all drawings bear all reference signs. Instead, in some of the drawings, some of the reference signs have been omitted for brevity and ease of presentation. Embodiments of the present invention are described below with reference to the accompanying drawings.

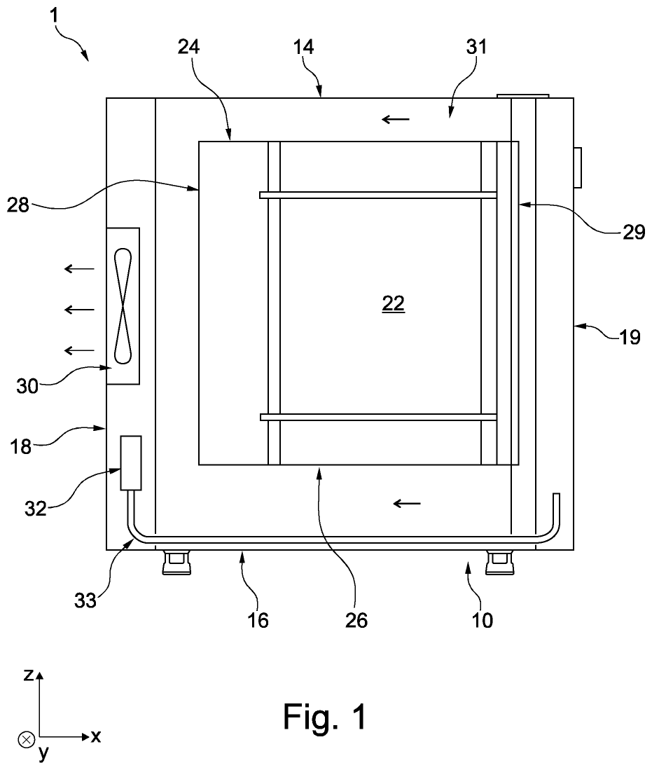

FIG. 1 is a schematic representation of an embodiment of the laboratory device 1 according to an embodiment of the present invention. The laboratory device 1 comprises an outer housing 10 which has a side housing 12, a rear housing 18, a ceiling housing 14, a lower housing 16, and a door housing 19. The rear housing 18 and the door housing 19 are at opposite ends of the laboratory device 1. The laboratory device 1 comprises an apparatus for generating the pressure difference, which apparatus has a fan 30. The laboratory device 1 further comprises an inner housing 20 which has side walls 22, a rear wall 28, a lower wall 26, a ceiling wall 24, and a door portion 29. The inner housing 20 can thus in particular define an inner chamber, for example an incubation chamber.

An intermediate region is defined between the outer housing 10 and the inner housing 20, which intermediate region comprises a rear region (between rear housing 18 and rear wall), side regions (between side housing 12 and side walls 22), and a door region between the door housing 19 and the door portion 29.

The fan 30 is arranged in the rear region so that air is drawn from the outer housing into the laboratory atmosphere to create a negative pressure within the outer housing (and in particular within the intermediate region). The apparatus for generating the pressure difference, in particular the fan 30, generates a flow in the direction of the apparatus for generating the pressure difference, in particular in the direction of the rear region. This flow is also present in particular in the portion between the lower housing 16 and the lower wall 26, which portion is referred to as the lower region, and in the portion between the ceiling housing 14 and the ceiling wall 24, which portion is referred to as the ceiling region.

The flow of air generated can transport particles, which are detached in the interior of the device, in the direction of the apparatus for generating the pressure difference. The door region may be fluidically connected to the rear region, the lower region, the ceiling region, and/or the side region. The connection can also exist in the open state of the laboratory device 1. A thermal insulation component can be arranged in the interior 31 of the device and in particular can fill the gap between the outer housing and the inner housing. Incomplete filling of the volume is advantageous in this case, so that air ducts are formed between the surfaces of the housing and the thermal insulation component, which air ducts are fluidically coupled to the apparatus for generating the differential pressure. For this purpose, in particular, openings are provided between the different regions of the outer housing. The flow of air directed from the apparatus for generating the differential pressure in the direction of the laboratory atmosphere can be passed through a filter.

The inner housing 20 can have a frame for receiving sample holders, in particular for receiving tray inserts, which are preferably made of metal.

The rear region can form a switch box which is separated from the interior of the device by a further wall. A flow of air from the interior of the device into the switch box can be realized through openings in the further wall. The apparatus for generating the pressure difference is arranged at least in the form of the fan in the switch box.

The apparatus for generating the pressure difference can comprise a pump 32 which can be designed in particular as a negative pressure pump or as a vacuum pump. The pump 32 can be arranged in the control cabinet and is fluidically coupled to the fan. This coupling can be realized, for example, by a pump outlet which is directed into the control cabinet, so that exhaust air from the pump 32 can be transported through the filter by means of the fan.

The pump 32 can be connected on the inlet side to a hose 33 which is at least partially arranged in the interior 31 of the device. The hose can be routed into the door region in order to generate a negative pressure in the door region and/or to generate or increase a flow of air for sucking particles out of the door region. In particular during a heating phase or sterilization phase of an incubator, the door region can have increased particle emission, with the particles being able to be transported away through the hose 33. At the connection of the lower housing 16 and the door housing 19, a connecting portion can be provided which fluidically connects the corresponding regions. The hose 33 can also be arranged in the connecting part. Furthermore, the connecting portion may be crescent-shaped, and an interior space of the connecting portion may be sealed against the door housing 19 and/or the lower housing 16 to establish flow communication between the door housing 19 and the lower housing 16 via the connecting portion.

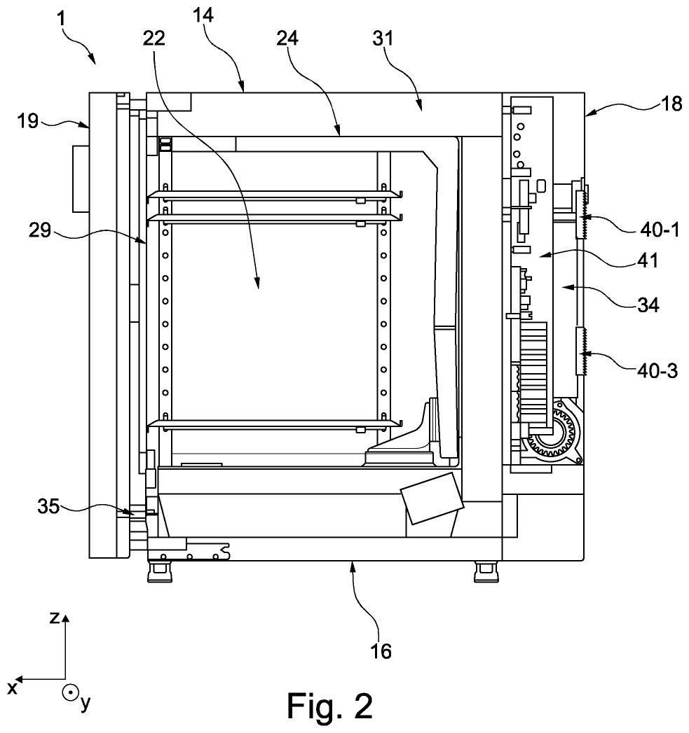

FIG. 2 shows a further schematic cross-sectional view of the laboratory device 1, with the door being arranged on the left in this view; this view is accordingly rotated by 180° around the z-axis in relation to the view in FIG. 1. A housing compartment 34 is provided in the rear housing 18 of the laboratory device 1, which housing compartment has a permanent exhaust air outlet. The filter can be arranged at the exhaust air outlet. Furthermore, an air-filled closing apparatus 35 is arranged on the door housing 19. The door housing can be mechanically locked to the lower housing 16 with the closing apparatus 35 in order to prevent the laboratory device 1 from being opened unintentionally. Furthermore, the closing apparatus can be locked to prevent unauthorized access to the interior of the laboratory device. With a closing of the closing apparatus 35, the seal 29 can be subjected to a static pressure. The lower housing 16 may be in the form of a double floor, with an outer wall being sealed against the laboratory atmosphere. A volume formed by the double floor can be fluidically coupled to the rear region or the switch box. A thermal insulation in the form of thermal insulation components can be arranged in the interior 31 of the device. The interior of the device can be—as already described—in particular fluidly coupled, so that gas can be conveyed from one region of the interior of the device to another region of the interior of the device. With a fluidic coupling, a flow of air can be generated in the interior of the device by means of the apparatus for generating the differential pressure, which apparatus is arranged in the switch box. At least one homogeneous pressure, in particular a negative pressure, can be achieved within the interior of the device with a fluid-technical coupling. The door may include a seal that seals the door relative to the side housing 12. The side wall 22 can have receptacles for tray inserts.

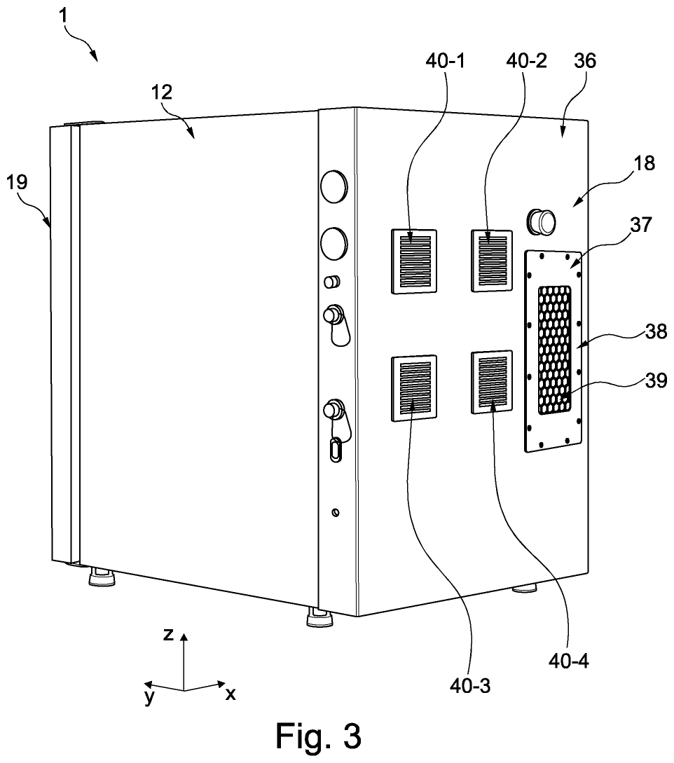

FIG. 3 shows a perspective rear view of an embodiment of the laboratory device 1. In particular, a filter holder 37 is arranged on the rear side 36 of the laboratory device 1 and is attached to the rear housing 18 by means of a releasable connection, in particular a plurality of screw connections. The filter holder 37 comprises a frame 38 on which the screw connections are arranged. Furthermore, the filter holder comprises a filter opening on which a protective screen 39 is arranged. The filter holder 37 can be placed on a housing surface of the rear housing 18. The filter 50 and/or the protective screen 39 may be offset inwardly from a surface of the filter holder 37. The protective screen 39 can have a periodic, in particular hexagonal screen web structure.

Furthermore, ventilation openings 40-1, 40-2, 40-3, 40-4 are provided on the housing surface of the rear housing 18, through which ventilation openings air can flow into the laboratory device, in particular into the rear housing 18, or the control cabinet. This flow of air can be used to cool electronic components inside the control cabinet. Laboratory air, which is sucked in via the ventilation openings 40-1, 40-2, 40-3, 40-4, can be filtered via the filter 50 and released into the laboratory atmosphere.

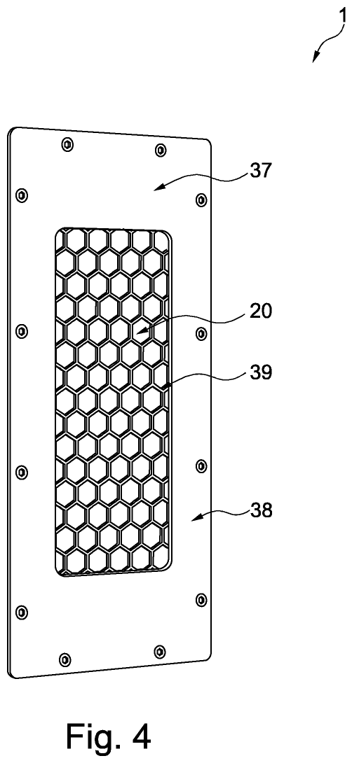

FIG. 4 shows a detail of an embodiment of the rear housing 18. The frame 38 of the filter holder 37 is attached to the rear housing 18 with a plurality of holding means, in particular screw or rivet connections. The filter holder 37 is designed to be releasable in order to be able to change the filter. The holding means can be arranged circumferentially on an edge of the filter holder 37. The holding means can be arranged substantially equidistantly with respect to a circumference of the filter holder 37.

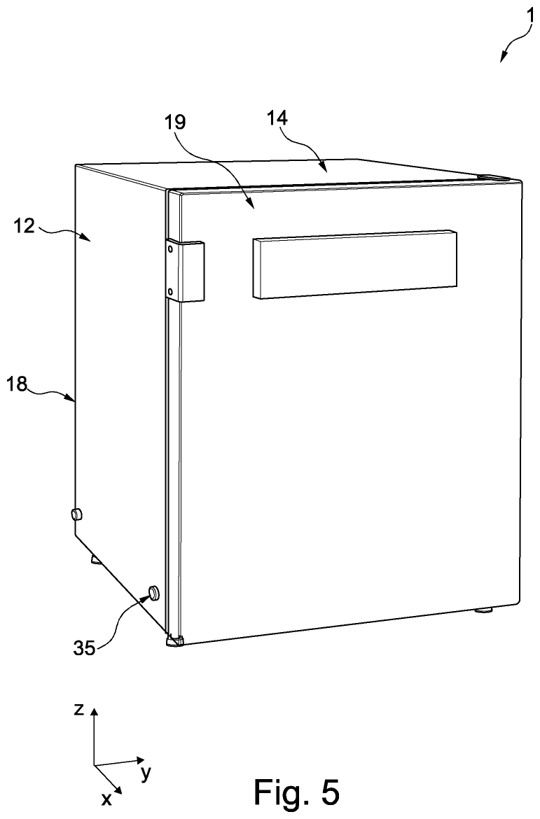

FIG. 5 shows a perspective front view of an embodiment of the laboratory device 1. The door housing 19 comprises a user interface with a display and input means. The closing apparatus 35 may include a lock cylinder having a keyhole arranged on the side housing 12. The closing apparatus 35 can be lockable via the keyhole, so that the door housing 19 cannot be opened in a closed state.

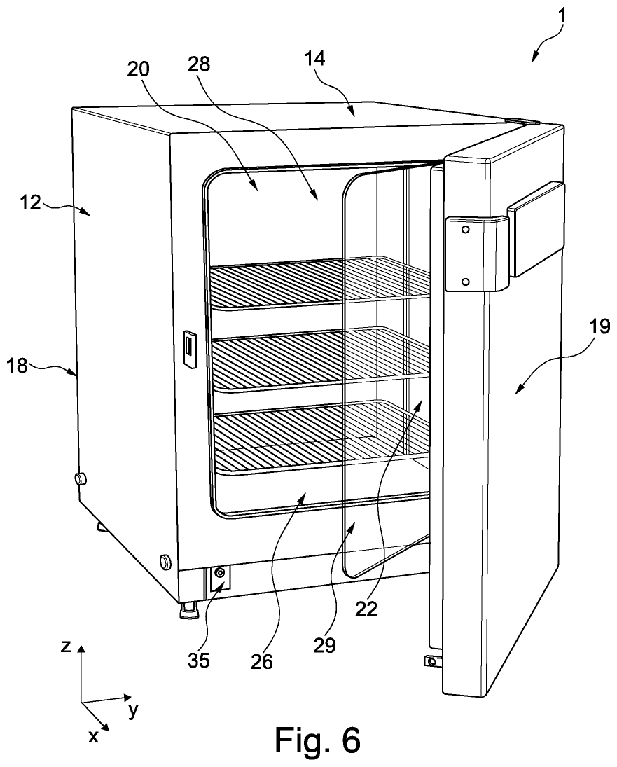

FIG. 6 is a perspective front view of an embodiment of the laboratory device 1. The door portion 29 comprises a glass door suitable for closing the inner housing 20. The glass door may abut a front device surface enclosing the ceiling housing 14, the lower housing 16, and/or the side housing 12. A seal can be arranged on the front surface of the device, against which the glass door can abut in a closed state, so that the glass door closes the inner housing 20 in a sealing manner.

The closing apparatus 35 may have a closing opening in the lower region, in which closing opening a pin of the closing apparatus, which is arranged on the door, can engage. With the door closed, the pin arranged in the closing opening can be locked. The lower portion can also have a separately attached housing part in which the closing opening can also be arranged. The side housing 12 can laterally close off the outer housing 10 at a full device height, with a lower housing 16 being arranged between the opposite side housings 12.

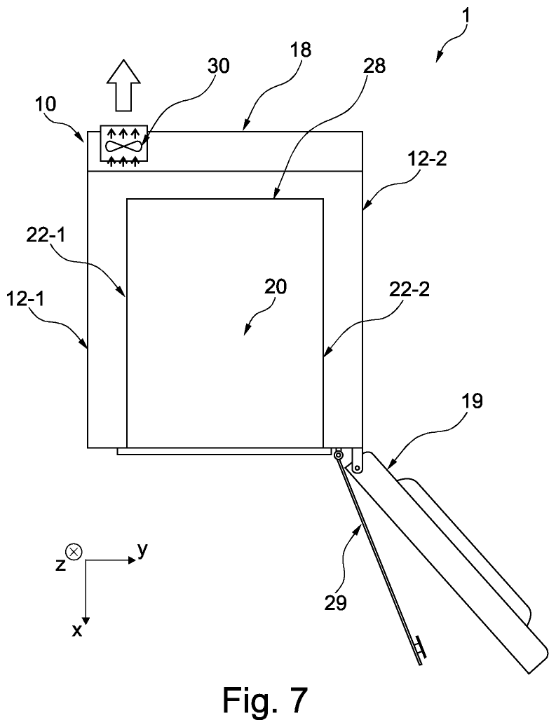

FIG. 7 shows a schematic horizontal cross-sectional view of an embodiment of the laboratory device 1 from above. The door is pivotally attached to the side housing 12 by a hanger apparatus to open the outer housing 10 and allow access to the inner housing 20. The intermediate region is between the inner housing 20 and the outer housing 10.

The regions in the intermediate space between the outer housing and the inner housing can be at least partially provided with insulation components, in particular with thermal insulating material. Accordingly, the fan 30 is fluidically connected to the intermediate compartment to direct particulate transport to a filter, which may be arranged downstream of the fan 30 with respect to the flow of air. As previously described, sub-regions (e.g., inside the door) can also be connected to a pump via hoses, so that particles can be transported to the pump via the hoses, and an outlet of the pump can be fluidically coupled to the fan.

Whenever a relative term such as “about,” “substantially,” or “approximately” is used in this document, that term is intended to include the exact term as well. In other words, for example, “substantially straight” should be construed to also include “(precisely) straight.”

While a preferred embodiment has been described above with reference to the drawings, a person skilled in the art will understand that this embodiment has been provided for illustrative purposes only and should in no way be construed as limiting the scope of the present invention which is defined by the claims.

What is claimed is:

1. A laboratory device, wherein the laboratory device is an incubator, comprising:
an outer housing which defines an interior of the device, wherein the laboratory device is designed to assume an operating state at which a pressure in the interior of the device is lower than an ambient pressure in the environment of the laboratory device.

2. The laboratory device according to claim 1, wherein the laboratory device has an apparatus for generating the pressure difference, wherein the apparatus for generating the pressure difference comprises a fan and/or a pump.

3. The laboratory device according to claim 2, wherein the apparatus for generating the pressure difference is designed to convey gas from the interior of the device into the environment of the laboratory device, wherein the laboratory device has a filter, which filter is arranged between an outlet of the apparatus for generating the pressure difference and the environment of the laboratory device.

4. The laboratory device according to claim 1, wherein the laboratory device has a total volume in the range of from 0.1 m³ to 2.5 m³.

5. The laboratory device according to claim 1, wherein the laboratory device has an inner housing which defines a chamber, wherein, in the operating state, the pressure in the interior of the device is present in a region that is delimited outside by the outer housing and inside by the inner housing, and wherein the pressure in the interior of the device is less than a pressure in the chamber.

6. The laboratory device according to claim 5, wherein the outer housing comprises:
a side housing;
a rear housing;
a ceiling housing;
a lower housing; and
a door housing,
wherein the rear housing and the door housing are arranged at opposite ends of the laboratory device,
wherein the inner housing comprises:
side walls;
a back wall;
a lower wall;
a ceiling wall; and
a door portion,
wherein the laboratory device is designed to generate a negative pressure in a door region which is delimited by the door housing and the door portion.

7. The laboratory device according to claim 2, wherein the laboratory device comprises at least one hose which fluidly connects the apparatus for generating the differential pressure to at least one other region.

8. The laboratory device according to claim 1,
wherein the laboratory device has an apparatus for generating the pressure difference, wherein the apparatus for generating the pressure difference comprises a fan and/or a pump,
wherein the laboratory device has an inner housing which defines a chamber, wherein, in the operating state, the pressure in the interior of the device is present in a region that is delimited outside by the outer housing and inside by the inner housing, and wherein the pressure in the interior of the device is less than a pressure in the chamber,
wherein the outer housing comprises:
a side housing;
a rear housing;
a ceiling housing;
a lower housing; and
a door housing,
wherein the rear housing and the door housing are arranged at opposite ends of the laboratory device,
wherein the inner housing comprises:
side walls;
a back wall;
a lower wall;
a ceiling wall; and
a door portion,
wherein the laboratory device is designed to generate a negative pressure in a door region which is delimited by the door housing and the door portion,
wherein the laboratory device comprises at least one hose which fluidly connects the apparatus for generating the differential pressure to at least one other region, and
wherein the at least one hose fluidly connects the door region and the apparatus for generating the differential pressure.

9. The laboratory device according to claim 7, wherein the at least one hose is made of a material that has a temperature resistance of up to at least 200° C., preferably up to at least 220° C.

10. The laboratory device according to claim 1, comprising at least one thermal insulation component, wherein the at least one thermal insulation component has a final layer which seals the at least one thermal insulation component, wherein the final layer comprises a film, wherein the film has a temperature resistance of up to at least 200° C., preferably up to at least 220° C.

11. The laboratory device according to claim 2, comprising a control unit which is designed to adapt a conveying capacity of the apparatus for generating the pressure difference to an operating mode of the laboratory device, wherein the control unit has a first operating mode, and the apparatus for generating the differential pressure comprises a fan and a pump, wherein only the fan is active in the first operating mode, wherein the control unit has a second operating mode, wherein both the fan and the pump are active in the second operating mode, wherein the control unit is designed to switch from the first operating mode to the second operating mode when a temperature limit value is reached.

12. (canceled)

13. The laboratory device according to claim 1, wherein the laboratory device has a total volume in the range of from 0.2 m³ to 1.0 m³.

14. The laboratory device according to claim 1, wherein the laboratory device has a total volume in the range of from 0.4 m³ to 0.8 m³.
